(19) United States
(12) Patent Application Publication
(10) Pub. No.: US 2021/0298711 A1
(43) Pub. Date: Sep. 30, 2021

(54) AUDIO BIOMARKER FOR VIRTUAL LUNG FUNCTION ASSESSMENT AND AUSCULTATION

(71) Applicant: Applications Technology (AppTek), LLC, McLean, VA (US)

(72) Inventors: Shahnaz MIRI, Washington, DC (US); Yasar Torres YAGHI, Arlington, VA (US); Fernando PAGAN, McLean, VA (US); Mudar YAGHI, McLean, VA (US); Sanjeev KHUDANPUR, Baltimore, MD (US); Jan TRMAL, Baltimore, MD (US); Hassan SAWAF, Los Gatos, CA (US); Jintao JIANG, Great Falls, VA (US); Mazda EBRAHIMI, Gerrardstown, WV (US)

(73) Assignee: Applications Technology (AppTek), LLC, McLean, VA (US)

(21) Appl. No.: 17/212,919

(22) Filed: Mar. 25, 2021

Related U.S. Application Data
(60) Provisional application No. 62/994,767, filed on Mar. 25, 2020.

(51) Int. Cl. A61B 7/00; A61B 5/00; A61B 5/08; G16H 50/30; G16H 50/80

(52) U.S. Cl. CPC A61B 7/003; A61B 5/741; A61B 5/6898; A61B 5/7475; G16H 50/80; A61B 5/0823; A61B 5/7415; G16H 50/30; A61B 5/7264

(57) ABSTRACT

A mobile device application prompts and conducts audio and/or video tests using a microphone on a smartphone, tablet or laptop in order to record and analyze a patient's speech, cough, breathing and other sounds in order to diagnose the patient with Covid 19, another ailment, or as having normal ranges not indicative of disease. The mobile device's tests and protocols use program instructions, AI processing and other automated tools to facilitate the speed and reliability of the testing.

### CROSS REFERENCE TO RELATED APPLICATIONS

The present application incorporates by reference herein, and claims priority to, prior U.S. Provisional Patent Application No. 62/994,767, filed on Mar. 25, 2020 entitled “Audio Biomarker For Virtual Lung Function Assessment And Auscultation.”

### FIELD OF THE INVENTION

The present invention relates generally to spectral analysis and, more particularly, to using mainly voice and respiratory spectral analysis for Virtual Lung Function Assessment and Auscultation (VLFAA).

### BACKGROUND OF THE INVENTION

The novel Coronavirus disease 2019 (COVID-19) is rapidly spreading infectious illness throughout the world, with a huge burden on healthcare systems, government, and nations worldwide. It has been suggested that about 80% of people with COVID-19 are asymptomatic or have a mild disease. However, about 20% may require higher level of care, of which 6% may be critically ill. Uncertainty about screening protocols has created a panic in population who demand having a “genetic testing” for the disease.

Screening suspected cases with real-time reverse-transcription-polymerase-chain-reaction (RT-PCR) assays is not practical due to high demand and limited resources for testing. Furthermore, the RT-PCR test has a high false negative rate (up to 40%) and it does not change the practice and management course of high-risk patients.

According to current guidelines released by CDC and WHO, people who have history of fever (or temperature above 37.3 degree), or cough and had exposure to confirmed COVID-19 cases or traveled to an area with confirmed cases within the past 14 days of those symptoms, are considered high risk. These cases should be placed in isolation to minimize the spread of the infection to others in contact with them. The key step for further investigation of these patients include evidence of shortness of breath, increased respiratory rate, or hypoxia (blood oxygen saturation <93%), at which time supportive care in hospital setting is warranted.

Due to limited testing resources, uncertainty about screening algorithm, and unavailability of remote clinical monitoring, general populations all over the world are in panic and this increases burden on healthcare systems. Screening breathing status of people with COVID-19 or those high-risk cases has created a significant burden on healthcare system and economy.

In this application, a system of a virtual audio biomarker powered by artificial intelligence is proposed for Virtual Lung Function Assessment and Auscultation (VLFAA) in asymptomatic cases in quarantine, patients with mild COVID-19, or those with high risk in close contact with known patients. Using artificial intelligence and a multimodal approach with the application program for testing for a specific disease, such as COVID-19 or a telemedicine application with this feature, screening and remote monitoring of cases will be simple and highly scalable. This tool can aid healthcare workers with providing additional objective data on respiratory status of cases via telemedicine. Healthy people can use this tool to have a baseline assessment of their respiratory function to re-assess any change in their respiratory function after potential exposure with COVID-19.

Although the application is initially designed to address the significant VLFAA in the COVID-19 pandemic, it can be extended to any other conditions that cause lung involvement as the underlying tests and multimodal approach can apply to all cases. One specific case is to help physicians to assess lung function through telemedicine in the future.

### SUMMARY OF THE INVENTION

According to an embodiment of the invention, spectral analysis and, more particularly voice and respiratory spectral analysis are analyzed in a software application for a mobile phone or similar device in order to perform a Virtual Lung Function Assessment and Auscultation (VLFAA).

According to an embodiment, the invention includes a mobile/web app user interface to collect data from user, machine learning components to model and analyze the data, and a reporting and summarizing component to aid the user to better perform self-evaluation and communicate with doctors. In the design, a multimodal approach is utilized to include additional measures (e.g., heartrate) to assist the assessment. The initial focus of the application is to provide VLFAA to reduce the risk of in-office visitation and the overburden of the healthcare system in the COVID-19 pandemic. Later, the application can be extended for VLFAA for any other conditions that cause lung involvement in different scenarios (e.g., telemedicine for lung function, evaluation of lung treatment, self-evaluation of lung function, etc.). The Machine learning technology includes Automatic Speech Recognition (ASR), Voice Activity Detection (VAD), Sentiment Analyses, and Speech Analyses.

According to an embodiment, a mobile device for diagnosing respiratory disease comprises a memory, a microphone and speaker, a display and a speaker. The memory stores at least one application program capable of executing a plurality of tests each having a test protocol, the application program including program instructions. The microphone and speaker receive audio and play audio respectively for a patient as part of test procedures. The processor is coupled to the memory, the display, the microphone and the speaker and is configured to execute the program instructions in order to: (i) instruct the user to perform an action including at least breathing, coughing, and reading text; (ii) activating the microphone to record the user following each instruction for each of the plurality of test protocols in respective recordings; (iii) analyzing and scoring the audio as according to criteria specific to each test protocol that are based on spectral data associated with the respective recordings; (iv) determining a composite score for presence or absence of the disease based on the individual scores; (v) and storing data, the test scores and the composite scores. The mobile device may be configured to instruct the user to perform each test according to its respective test protocol using the display and/or speaker of the mobile device.

### BRIEF DESCRIPTION OF THE FIGURES

The features and advantages described above and elsewhere of the present invention will be more fully appreciated with respect to the appended figures, which are described below.

FIG. 1 depicts a block diagram of a smartphone and a laptop interacting with a person who is speaking to collect audio and process the audio with AI models according an embodiment of the present invention.

FIG. 2 depicts an illustrative spectrogram of the voice from of a patient with a potential COVID-19 condition, showing a total duration 20 seconds for the patient versus about 27 seconds for a control, with an increase in voice sound resonance in the patient compared to a control case.

FIG. 3 depicts an illustrative speech spectral analyses on one-minute speech from a patient with a potential COVID-19 pneumonia diagnosis, showing frequent pause and breathing during one-minute of talking, with B indicating breathing and an audio sampling frequency of 16 KHz.

FIG. 4 illustratively depicts a voice spectrogram after pneumonia treatment and recovery, showing decreased speech interruption with breathing during one minute of talking and decreased high frequency breath sounds compared to the initial evaluation.

FIG. 5 illustratively depicts a breathing sound spectrum during deep inhalation and exhalation in a patient with a potential COVID-19 pneumonia diagnosis showing successive days over 13 days from an illustrative diagnosis to recovery.

FIG. 6 depicts an illustrative recording of left and right lung sounds in normal lungs while a patient speaks /E/ and /A/.

FIG. 7 depicts an illustrative recording of left and right lung sounds in lungs of a patent illustratively diagnosed with Covid-19 pneumonia while a patient speaks /E/ and /A/.

FIG. 8 depicts an illustrative comparison of an intentional cough in a patent with COVID-19 as compared to an illustrative normal patient control.

FIG. 9 depicts a method of performing disease testing for a respiratory illness such as COVID-19 and reporting of results and raw data according to an embodiment of the present invention.

FIG. 10 depicts an illustrative block diagram of a mobile device and its interaction with a network to facilitate the testing of a user for Covid-19 or other respiratory illness, according to an embodiment of the present invention.

### DETAILED DESCRIPTION

According to an embodiment of the invention, spectral analysis and, more particularly voice and respiratory spectral analysis are analyzed in a software application for a mobile phone or similar device in order to perform a Virtual Lung Function Assessment and Auscultation (VLFAA). According to an embodiment, the invention includes a mobile/web app user interface to collect data from user, machine learning components to model and analyze the data, and a reporting and summarizing component to aid the user to better perform self-evaluation and communicate with doctors.

### COVID-19 Relevance

In the following description, COVID-19 is used as an example/motivation to design the application and tests. However, the application itself can and will be extended to other conditions that cause lung involvement.

### System Design

The following describes a new approach to use a voice and heartrate biomarker tool VLFAA in healthy people, high risk cases, and patients with COVID-19. The system consists of four components:
(a) A Smartphone/web application as a user-interface to collect voice data and heartrate from user and interact with users.
(b) A Smartphone/web application as a user-interface to label/tag data based on medical doctor's guideline.
(c) A battery of tests that are made virtual (remotely) and automatic with machine learning and voice analyses.
(d) A risk scoring and report system.

### A Smartphone/Web Based User-Interface

A novel Smartphone/web application for screening and monitoring of respiratory status in cases with COVID-19 is proposed. The Smartphone/web application is compatible with Windows, iOS, and Android with a temporary access to the device microphone for voice recording at the time of use. The application also acquires heartrate data for multimodal analyses. Chatbot with the same function can be available via different online platforms including WhatsApp and Facebook. The application will direct the user to perform a battery of tasks.

A single page mobile friendly web application is developed for the data collection. The application will utilize single sign-on to identify the user for longitudinal analysis of results.

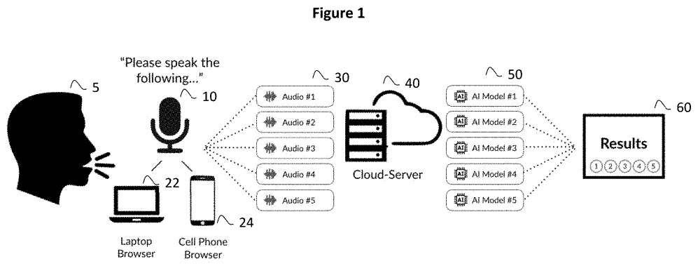

FIG. 1 depicts an illustrative block diagram of a system in which an embodiment of the present invention may find application. Referring to FIG. 1, a person is in proximity to a laptop 22 or mobile phone 24 that may be running an application or a browser program that presents a user with an interface that permits the user to perform speaking tests and make sounds such as breathing and coughing into a microphone 10. The laptop or mobile device 22 or 24 make requests of the user to produce certain types of sound and record audio associated with the user and the specific sounds that the application requests from the user. Illustratively the recordings 30 may be stored in any processable format. Once recorded, the audio 30 may be processed on the laptop 22, mobile device 24 or by a cloud server 40. The processing may be done according to different AI models 50 associated with different test and processing frameworks. In 60, the results including final results and intermediate results may be stored for later processing and presentment to a physician and/or a user or patient.

Referring to FIG. 1, tests are driven by a laptop 22 or mobile device 24 and start with basic screening questions, followed by a five-step lung function assessment. Subjects will be instructed to stay in a quiet place and keep the Smartphone/microphone at around eight inches from the mouth. Each step of the virtual lung function assessment test includes short concise instructions and record the patient's audio for analysis. The user will have the option of continuing to the next step or re-recording the audio. Upon completion of each step, the recorded audio will be submitted to the cloud service for analysis.

At the end of the cough pattern recognition (final) test, the user is presented with an opportunity to record additional comments regarding their condition. This audio is be analyzed, transcribed, and mined for additional metadata. Once finished, the user is presented with a single page test result page.

The data collection and processing systems can be adopted to run in a HIPPA compliant environment if necessary.

### A Smartphone/Web Based User Interface for Data Annotation

Machine learning applications require high-quality labeled data. For this application, the data annotation quality is of especially important due to its medical relevance. Towards this end, medical doctors will actively participate in designing the data annotation guideline. The user interface will be designed user friendly for compliance with the guideline.

### Voice-Based Tests

In this section, we describe the five clinically relevant tests of the subject's respiratory health status. We developed recognition algorithms from recorded respiratory sounds near the mouth and the subject's heartrate data using a Smartphone/web application. Based on reported symptoms of participants (experiencing respiratory symptoms of COVID19 vs. asymptomatic), recordings will be classified with a two-phase algorithm (signal analysis and pattern classifier using machine learning algorithms). Each audio recording together with the heartrate data is passed to the appropriate analytic model in an auto-scaling server environment. Each of the five analytics models are designed to accept an audio file and heartrate data and return the results for that test. Results from every step, the pre-evaluation questions and transcription of additional audio comments are stored in a database.

The tests will be performed automatically with the aid of machine learning technologies (ASR, VAD, and Speech Spectral Analyses). Specifically, VAD events include hesitation, breath, laughter, cough, applause, click, beep, ring, cry, clapping, lip-smack, clearing-throat, sneeze, whisper, sigh, music, noise. In this application, VAD has three functions: (a) to automatically detect end of speech to stop the test, (b) to detect breathing and cough sounds, and (c) to detect other non-speech sound (sneezing, clearing-throat, etc.). A sample of 100 control cases will perform the same task to produce normal range values. The results from all other cases will be reported compared to normal values.

Voice Analysis and Voice Spectral Analyses is a common technology used here across all tests that include spectrogram, spectral power spectral density (PSD), voice intensity, pitch, formants, linear predictive coding (LPC). A spectrogram is a visual representation of the short-time spectrum of frequencies along the time axis. PSD is the measure of signal's power/energy at each frequency slot. Voice intensity is the volume of the sound. Pitch is the glottal vibration frequency. Formants are the vocal tract resonance frequencies. LPC is a method used to extract the spectral envelope of a voice signal, especially for vowels. In this application, LPC can be used to estimate formants and their intensity that are highly related to the subject's vocal tract shape and configuration and thus indirectly reflect the person's lung condition.

A multimodal approach is implemented here to include the subject's heartrate acquisition. With Smartphones, there are two approaches to acquire subject's heartrate data: (a) the heartrate can be estimated from the subject's voice or breathing sound and (b) the heartrate can also be estimated using the device's camera. In both cases, there are existing open-source algorithms to for the estimation. In each test that is described below, the heartrate will be acquired at the beginning of the test and at the end of the test with both approaches (voice-based and image-based). The heartrate data, together with the voice spectral data, are used to aid the assessment of the lung functions.

Test-1: Lung Reserve Assessment with Counting

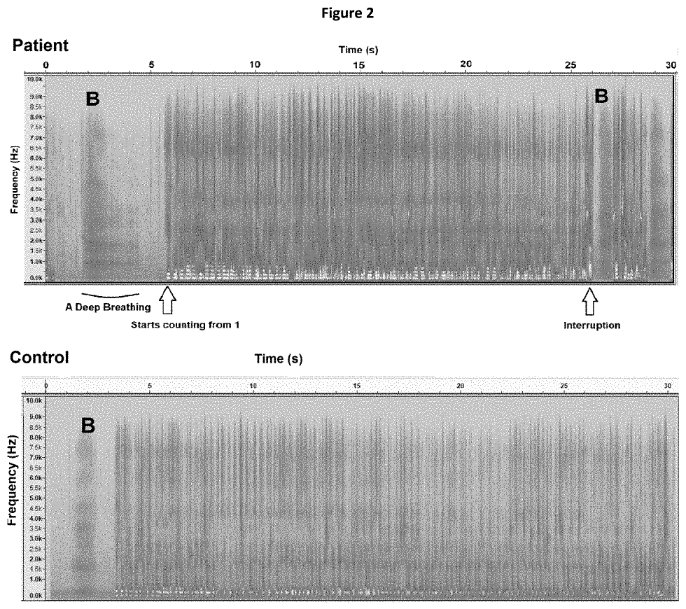

In this test, the subject presses a button to start the test. He/she takes a deep breath as much as possible and then start counting 1, 2, 3, 4, . . . , without taking any breath in between. The clinical goal is to determine the subject's lung capacity and ability to exhale in a controlled manner. This measurement correlates well with Vital Capacity and Negative Inspiratory Force (NIF) which can be severely reduced in a patient with pneumonia and COVID-19. This measurement can also be used in neurological patients that have compromised pulmonary function. A useful metric in determining this capacity/ability is to record the highest count the subject reached, as well as the total duration for which the subject was able to sustain the counting. The duration of uninterrupted counting (seconds) is an indicator of respiratory reserve (the longer, the more reserve, the less likelihood of pneumonia or lung involvement) (see FIG. 2). In addition, the voice PSD during counting and the subject's heart rate are also examined.

This will be achieved by state-of-the-art ASR technology. Our standard deep-neural network based acoustic models are trained on tens of thousands of transcribed speech data. Furthermore, a constrained language model will be developed with the nominal count sequence (i.e., one, two, three, four, . . . ) as the most likely transcript, but also permitting common disfluencies (e.g. ‘uh’ and ‘um’, number fragments, skipping of numbers).

The outcome of the automatic transcription will be both the identity of the highest count reached by the test subject and the duration of the utterance. Both are standard output of the ASR engine, and a post-hoc alignment of the recorded speech with the transcripts may be used to further refine the timestamps. In addition, the word confidence scores, which are also standard output of the ASR engine, can be used to assess the clearness of the speech. The ASR systems are available in many different languages, including English, Spanish, Mandarin, Italian, Arabic, Farsi, and other languages that the test subject may be comfortable counting in. This test therefore can be made multilingual to facilitate universal access (and the same for the following tests).

Additional analyses will be made available when (1) medical doctors identify other specific kinds of disfluencies that individuals suffering from respiratory distress may exhibit and (2) we use machine learning technologies to identify clinically relevant factors on transcribed data from patients known to be distressed and contrasting data from healthy readers.

Accordingly, the test protocol may include:

1. Instruct the patient to take a deep breath and then begin counting;

2. Begin audio and or video;

3. Collect and store raw data, such as shown in FIG. 2, data collection from the user during the user speaking the count;

4. Compare audio signature of an average control or a prior recording of the healthy user doing the test; and

5. Score the test as more or less probably for the presence of the disease such as COVID-19.

For example, When the PSD shows frequent interruptions for breaths or shows higher spectral power levels at higher frequencies than the control, this indicates a higher likelihood of the COVID-19 disease and the scoring will reflect that as a higher or lower score according to a chosen ordinal numerical scale or a color based scale, for example, green, yellow, red.

### Test 2: Speech Assessment

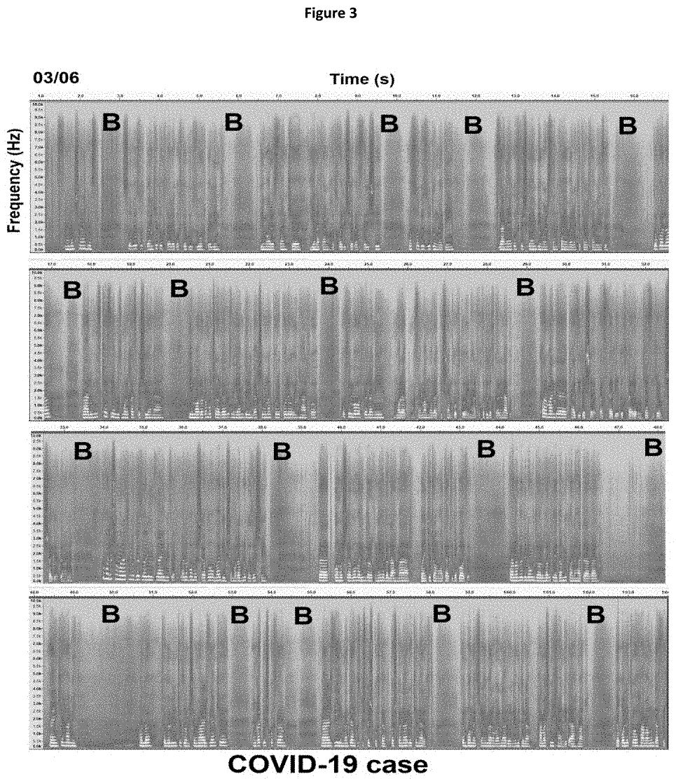

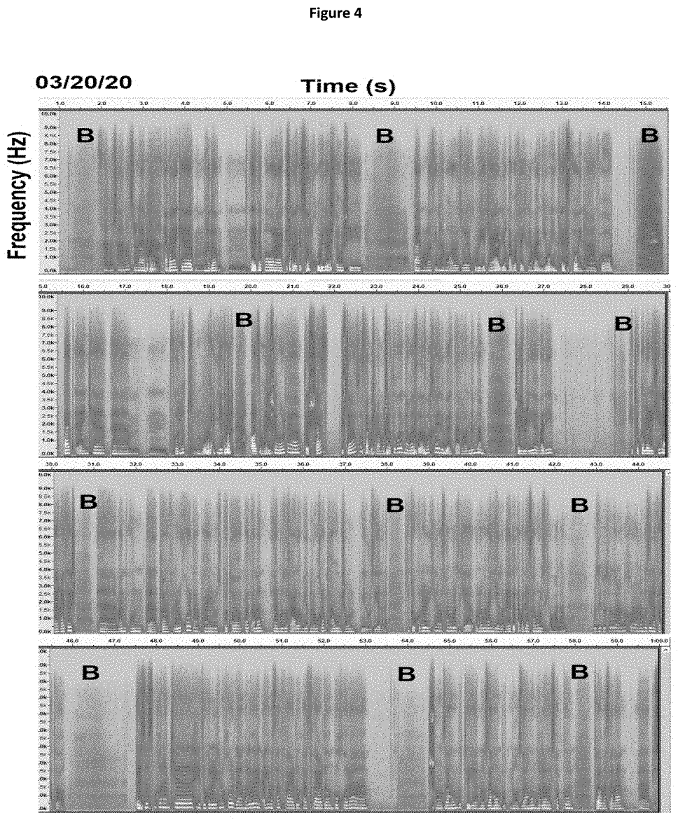

In this test, the subject will be presented with a paragraph and press a button to start. He/she is instructed to read the text loudly, fluently, and with minimum pause and breathe normally. He/she will keep reading as long as he/she can. After reaching the end of paragraph, he/she can go to the beginning of the paragraph. The clinical goal is to determine the subject's speaking rate and vocal effort while reading. A useful metric in determining this is to measure the syllables per second spoken in the beginning, middle and end of the reading—patients having respiratory distress will likely slow down—and have changes in the spectrogram, PSD, pitch, and formants in vocalic segments. Another useful metric is the number of breaths per minute that a subject takes while reading the passage, with 15-20 breaths per minute (as opposed to 10-12 for healthy subjects) are indicative of respiratory distress (see FIGS. 3 and 4). In addition, the subject's heartrate data acquired in the beginning and end of the test are also analyzed for correlations.

Again, ASR will be used in this test. Furthermore, a constrained language model will be developed with the reading prompt as the most likely transcript, but also permitting common disfluencies (e.g. ‘uh’ and ‘um’, false starts, repetitions of phrases, skipping of an occasional phrase, switching of adjacent words). An automatic alignment of the recorded speech with the ASR transcript—expanded to phonetic-level details—will provide phoneme-level timestamps, inter-word pause durations and other metrics necessary to compute local speaking rate. It will also identify vocalic segments from which speech spectral energy in total and in different bands will be calculated and displayed as a function of speaking time.

An interesting respiratory artifact that is usually ignored (intentionally) by ASR systems, but which may be of key significance in this setting, is accurately annotating the transcript with the location of breaths taken—primarily inhalation—by the subject while reading the passage. This is traditionally not considered a part of the transcript and is therefore not present in the output of the ASR engine. Therefore, one novelty from this application is that we develop techniques for detecting this respiratory event by augmenting the ASR engine's acoustic models.

We conjecture that inhalation events happen between words, or by the insertion of a pause between two syllables within a word. Furthermore, between-word inhalations are actually (already) captured by the ASR engine, in the form of “optional silences,” while within-word pauses are absorbed as a part of the previous phoneme, giving it an unusually long duration. As such, we design a two-pronged strategy to detect breath (inhalation) locations in these recordings.

Intra-word breath. We will modify our pronunciation lexicon to permit “optional silence” between syllables of a word, and extract their presence form an automatic (forced) alignment of the automatic transcript with the acoustic recording. Once such silence segments are detected, the procedure described below for inter-word silence will be applied. For this purpose, we segment word pronunciations into syllables or perform the morphology analyses.

Inter-word breath. We will use an annotated corpus of breathing sounds to classify each hypothesized (optional) silence segment between speech segments as either inhalation of other sounds. We expect that a deep neural network trained with input features such as spectral entropy, relative spectral weight between different regions of the spectrum, and the log spectrum itself will be able to perform the task.

This “clinically augmented” ASR engine will enhance the speaking rate and vocal effort measurements through the time-course of the reading, with the average breathing rate of the test subject.

Accordingly, the test protocol may include:

1. Instruct the patient to take a deep breath and then read text presented to the user in a passage via an application program, such as a mobile phone app. The text may be presented in the application or via text message for example;

2. Begin audio and/or video;

3. Collect and store raw data, such as shown in FIG. 3, of the user during the user speaking the text;

4. Compare audio signature of an average control or a prior recording of the healthy user doing the test; and

5. Score the test as more or less probably for the presence of the disease such as COVID-19.

For example, when the PSD shows frequent interruptions for breaths, shown in FIG. 3 with “B”, that may indicate the presence of the disease. Also, when the spectral analysis over time shows higher spectral power levels at higher frequencies than the control, this indicates a higher likelihood of the COVID-19 disease. In both cases, the scoring based on spectral analysis of where breaths occur and the spectral content will reflect that as a higher or lower score according to a chosen ordinal numerical scale or a color based scale, for example, green, yellow, red.

### Test 3: Deep Breathing Assessment

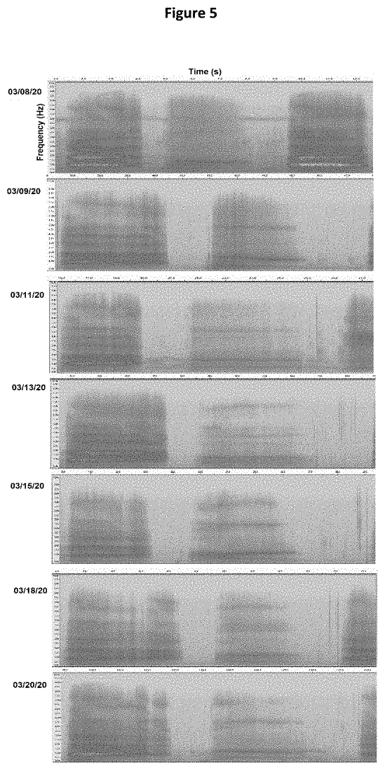

In this test, the subject presses a button to start the test. He/she will take a deep breath in, pause, and then exhale, repeating this step five times while recording his/her voice. The clinical goal of this test is to measure the duration, spectrogram, and PSD of in the high frequencies during inhalation and exhalation as a distressed lung will often lead to higher PSD in the high frequency regions (see FIG. 5). Specifically, the voice-based heartrate data can be estimated from the breathing sounds and thus adding more resolution.

For this test, we develop acoustic analysis techniques to automatically segment and label the audio recording with beginning and ending times of inhalation, holding of breath, and exhalation. Specifically, we believe that exhalation segments will be characterized by high energy in the upper spectrum, and boundaries of segments will be characterized by transients in the spectrum caused by the formation or release of the glottal stop.

Accordingly, the test protocol may include:

1. Instruct the patient to take five deep breaths, each followed by an exhale.

2. Begin audio and/or video;

3. Collect and store raw data, such as shown in FIG. 4, of the user during the user drawing breaths and exhaling;

4. Compare audio signature of an average control or a prior recording of the healthy user doing the test; and

5. Score the test as more or less probably for the presence of the disease such as COVID-19.

For example, when the PSD shows frequent breaths, shown in FIG. 4 with “B”, that may indicate shallow breathing and the presence of the disease. Also, when the spectral analysis over time shows higher spectral power levels at higher frequencies than the control, this indicates a higher likelihood of the COVID-19 disease. In both cases, the scoring based on spectral analysis of where breaths occur and the spectral content will reflect that as a higher or lower score according to a chosen ordinal numerical scale or a color based scale, for example, green, yellow, red. This test may be repeated over time as shown in FIG. 5, and such tests may reveal as shown deeper breaths over time, longer breath and exhale cycles over time, and fewer high frequency PSD compoents when a patient is recovering from a disease such as COVID-19.

### Test 4: Egophony Screening Test for Pneumonia

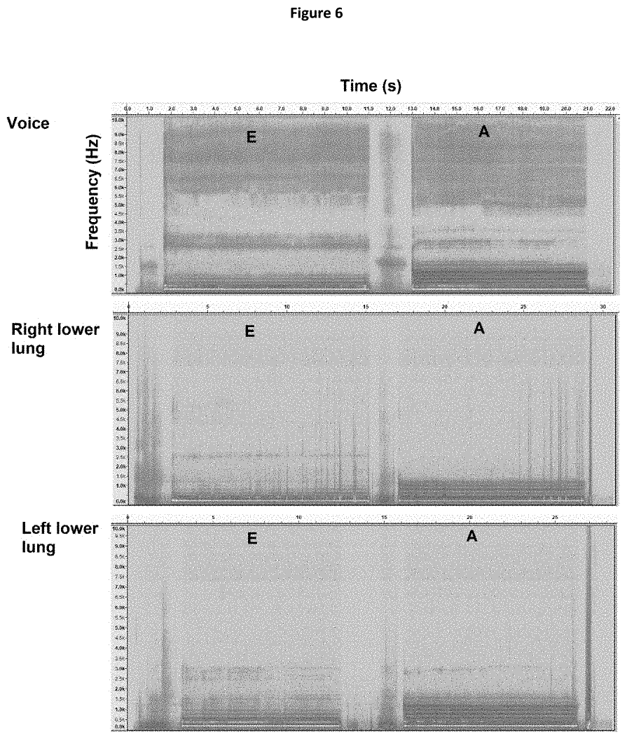

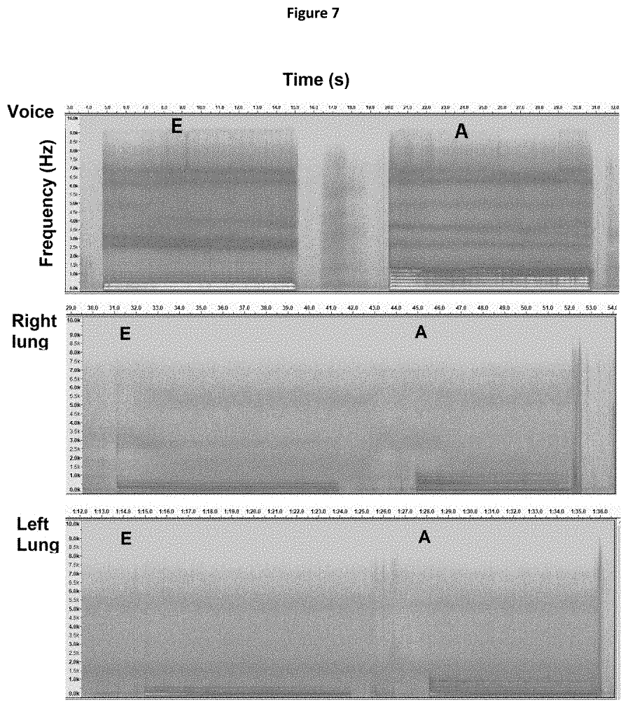

In this test, the subject presses a button to start the test. He/she takes a deep breath in, then says /E/ loudly without breathing in-between as long as he/she can. The subject then repeats the same process for /A/. Egophony is an increased resonance of voice sounds heard when auscultating the lungs, often caused by lung consolidation and fibrosis. It is usually due to enhanced transmission of high-frequency sound across fluid, such as in abnormal lung tissue, with lower frequencies filtered out. We have created a new method of Egophony assessment via voice analysis which enables virtual assessment and auscultation of cases suspected for pneumonia. The clinical goal is the measure the closeness between the sound of /E/ and /A/ from the subject in terms of spectrogram, PSD, formants, heartrate, and voice intensity. It has been shown that subjects with COVID-19 will pronounce their /E/ closer to their sound of /A/, although both sounds have an increased high frequency sounds (see FIGS. 6 and 7). Like Test-3, the voice-based heartrate data can be estimated from the /E/ and /A/ sounds and thus adding more resolution.

This test requires detecting the beginning and end times of the two segments, which can again be done using deep-neural network based acoustic models. We anticipate challenges for audio in which the subject's vocalization is interrupted by coughs or sneezes, or other disruptions that may be expected in unhealthy test takers. This will require creation of a flexible alignment model that nominally expects to hear an /E/ segment followed by an /A/ segment but permits commonly observed deviations.

Acoustic models for aligning /E/ and /A/ are robust and language independent. Therefore, the test can be administered to speakers of any language, enabling universal access.

Accordingly, the test protocol may include:

1. Instruct the patient to say and hold /E/ and separately /A/.

2. Begin audio and/or video;

3. Collect and store raw data, such as shown in FIG. 6, 7, of the user during the user saying /E/ and /A/;

4. Compare audio signature of an average control or a prior recording of the healthy user doing the test;

5. Compare audio signature of the /E/ to the /A/ and determine the extent to which the E is distinguishable from the /A/;

5. Score the test as more or less probably for the presence of the disease such as COVID-19.

For example, when the /E/ and /A/ sound the same, by for example an ASR program not being able to tell the difference between the E and the A, or when the ASR program can distinguish the /E/ and the /A/ , but the confidence score of one or both is very low, that indicates the presence of the disease. Also, when the spectral analysis over time shows higher spectral power levels at higher frequencies than a control, this indicates a higher likelihood of the COVID-19 disease. In both cases, the scoring based on ASR, including in some cases confidence levels, and based on the SPD at higher levels at higher frequencies as compared to a control, will reflect a higher likelihood of a disease such as COVID-19 and a higher or lower score according to a chosen ordinal numerical scale or a color based scale, for example, green, yellow, red. This test may be repeated over time as shown in FIG. 5.

### Test 5: Cough Pattern Recognition

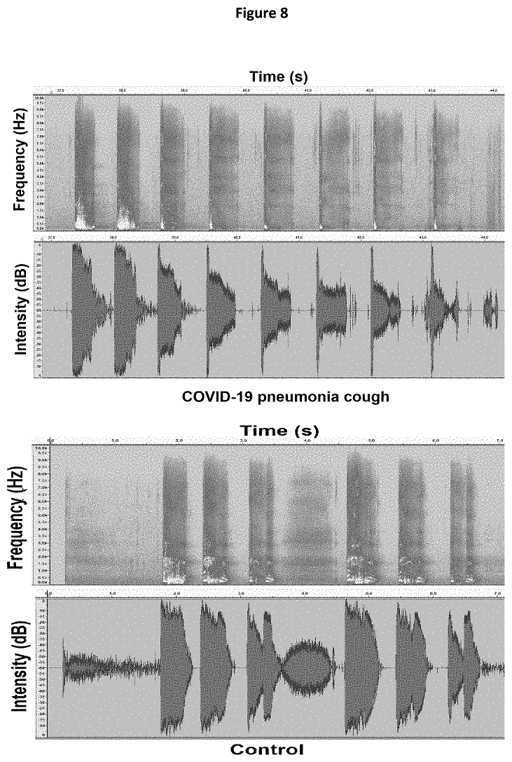

In this test, the subject presses a button to start the test. He/she tries to cough for at least ten seconds. The goal of this test is to evaluate the respiratory health status of the subject based on the cough pattern based on the number of coughs, sound waveform, PSD, and sound intensity (see FIG. 8).

As clinically annotated cough samples are available, machine learning techniques will be applied to automatically classify the coughs into those from healthy subjects versus those from subjects suffering from a few pathological lung conditions. Furthermore, machine learning will help us learn all the different types of coughs (e.g., wet cough and dry cough) and differences in frequency. One example is that cough in pneumonia will be different than in Asthma or bronchitis or Pertussis (whopping).

Accordingly, the test protocol may include:

1. Instruct the patient to cough.

2. Begin audio and/or video;

3. Collect and store raw data, such as shown in FIG. 8, of the user during the user coughing;

4. Compare audio signature to an average control or a prior recording of the healthy user doing the test; and

5. Score the test as more or less probably for the presence of the disease such as COVID-19.

For example, the cough test may reflect a gradually declining PSD signature when the disease is present as opposed to a more random but not declining PSD in a healthy control.

### After Test: Meta Data and Sentiment

After the tests, the subject can choose to opt in to provide meta data with speech. The subject is instructed to talk about his/her age, city, medical condition, medical history, and activities before the test. We will conduct sentiment analyses on the audio to predict the anxiety level of the subject.

### Scoring and Reporting

The system will compute a risk score for lung function from each test. In the end, an overall risk score is computed by weighing and summing the individual scores. The weights will be learned from the data using machine learning. A one-page report output of individual case analysis will be provided immediately after each test with explanation that consists of three sections:
(a) Summary Section: This section will provide a simple Red/Yellow/Green light (or similar) output to the user regarding their test results. This section will also include some links to resources or information to help the user stay safe. CDC recommendations etc.
(b) Detail Section—Users who wish to learn more about their results for specific tests will be able see their results and compare them to acceptable range.
(c) Trend Section—A histogram of test scores to help patients assess changes in their score over time.

Further interpretation of individual cases compared to normal range can be done via telemedicine visits.

There will be an export or email option, so the patient can easily share the data with their doctor.

### After COVID-19

As discussed earlier, the application may be designed with the motivation to address the virtual lung function assessment and auscultation (VLFAA) issue in the COVID-19 pandemic. After the crisis, the application may be used for VLFAA or for any other conditions that cause lung involvement, for example, to evaluate respiratory infections like flu, to find the cause of breathing problems, to diagnose and monitor chronic lung diseases (including asthma, allergies, and bronchitis), to access whether lung disease treatments are working, to check lung function before surgery, and so on. Therefore, data may be collected through the application to classify into different lung condition categories, per medical doctors' suggestions. Machine learning technologies will be applied to data from each condition and across conditions. The rationale is that the above-mentioned tests measure some of most important and fundamental items in the standard lung function test and can be used as an alternative to other lung function tests. As these are virtual lung function tests, they may be readily incorporated in clinical settings, clinical trials/research studies, and telemedicine cases. Besides, the application can serve as a data collection platform and share data and findings with medical doctors. At the same time, the application may be open to or used for new tests that medical doctors would recommend.

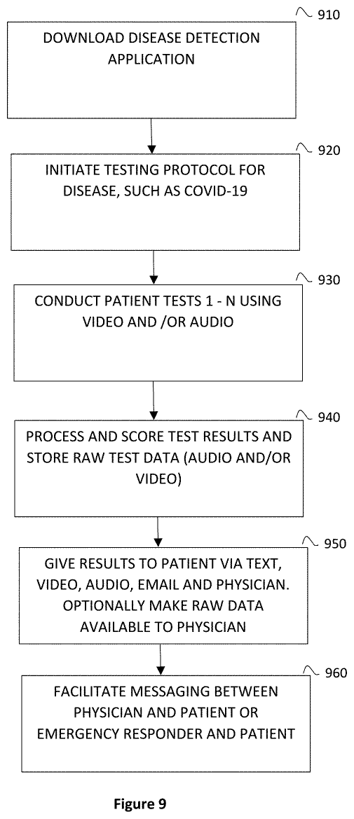

FIG. 9 depicts a method of using a mobile device equipped with testing applications and optionally a telemedicine application to facilitate testing a mobile phone user for Covid 19 or another respiratory disease. Referring to FIG. 9, a mobile App may be used according to some embodiments of the invention. The App may be distributed and downloaded for example as shown in step 910 from the Apple App Store or Google Play. It otherwise may be made available or distributed for use on mobile devices, like mobile phones, tablets or laptops, or other computing devices.

The App may have a user account management that communicates with a backend server that lets a user identify himself or herself to the testing application and server. Each user may have his/her own account. In this example, the user can perform a baseline evaluation prior to being sick. This application may be a test specific application such as a Covid-19 test application. Alternatively, it may be a Respiratory test application. Still further, the application may be a telemedicine application that not only conducts tests but that makes the testing interactive with the help of a physician and/or makes data from the testing available to a physician to review the test results and/or the raw data in order to participate in patient diagnosis.

Referring to FIG. 9, the user downloads the disease detection application to a mobile device, such as a mobile phone. The user then initiates a testing protocol for a disease, such as Covid-19 in step 920. The testing protocol may, for example, run the patient through a series of tests 1-5 as described above in step 930. As the tests are then conducted, they may include presenting audio queues to the user to cough, count, breathe, etc. via the speaker on the mobile device. Alternatively, the user may be prompted with text presented to the user via an in application presentation of the text or, alternatively, through the sending of message and command prompts to the user via text message or SMS.

During each test, the user is prompted to take some action and the microphone or video camera or both capture data from the user and process the data according to the techniques described above. For each test, as shown in 940, the raw data may be stored on the mobile device and/or a database accessible via a network for processing by the mobile device and/or a server. The raw data may also be made available to a physician via the network or by sharing the mobile device. Similarly, the raw data may be processed by the server or the mobile device according to the protocols described above in step 940 to determine a score associated with the disease being more or less likely. The tests are each scored individually and then in aggregate in order to determine a diagnosis, such as the presence or absence of a disease, such as Covid-19 or a likelihood of having the disease. The application may also report the results in step 950 in an order of significance for determining the presence or absence of the disease to facilitate review by the patient or a treating physician or emergency responder.

In 960, the results and raw data may be sent to the patient or a physician be text or by making the reported results and raw available via the testing application, via a telemedicine application, by text message or by other communication technique. By making the raw data available, such as video and/or audio, the physician or user may review not only the test results and the summary of the test results, but also view the actual tests in order to get additional information useful for treatment. Multiple tests may be taken over time. For example, a patient may perform the test in a good health state as a control to use as a baseline if the patient gets sick in the future. In addition, on the onset of symptoms, the patient can perform the test on successive days in order to determine if the disease, such as Covid-19 is getting better or worse and at what rate. Alternatively, the successive testing may be used to determine if the patient is getting better by trending toward having fewer symptoms.

The testing application or telemedicine application may also allow the physician to message the patient, annotate the case and share the case with other physicians or make the case notes available via the application available to other physicians or emergency responders. The patients data and all other patient's data may also be aggregated along with other information about the user collected form the mobile device's location or the as part of the account setup to determine whether there is an increase of symptoms of disease in a particular geographic location at a particular time. To facilitate this, the user or physician in the testing or telemedicine application may input some information about the user, including name, home address, work address, insurance information, employer and other information. The user may administer any tests him or herself or the testing may be facilitated by another person for the user.

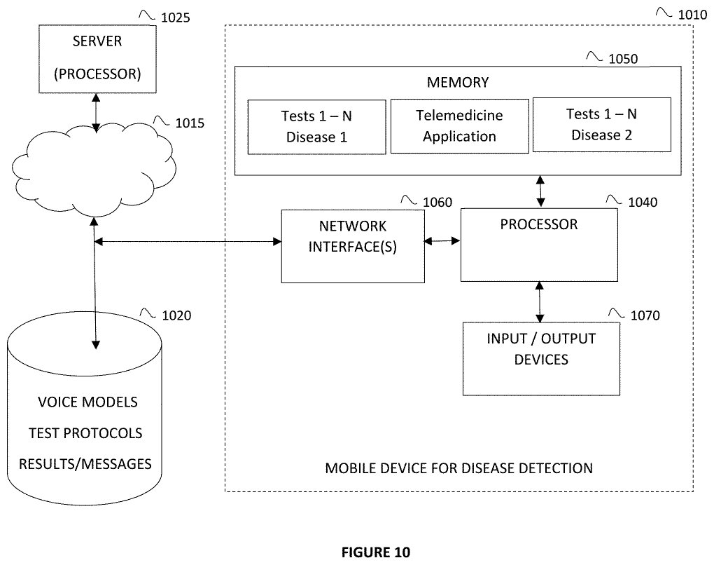

FIG. 10 despicts a mobile device 1010 coupled to a network 1015 such as a local area network, a wide area network or the Internet. The mobile device may be locally coupled to a database 1020 on the mobile device itself, or the database 1020 may be part of a local area network or may be accessible via the Internet or a wireless connection to a cloud based data platform. The mobile device may also access a server 1025 via a network connection that is able to perform the processing of the data collected and the testing described in this application.

The mobile device 1010 and the server 1025 each include a processor 1040, memory 1050, and networking interfaces or units 1060 that couple the mobile device 1010 and the server 1025 to networks, such as telephone and data networks. The mobile device 1010 and back end server are also each coupled to each other to exchange data. The memory 1050 stores program instructions as shown for tests 1-N for each disease for example, and for applications, and for other functional pieces that may be used to test and analyze patients speech, breathing and lung sounds. The memory may store, for example, disease testing protocols for tests 1-5 described herein for a respiratory illness such as covid-19, but also may store separate ones for asthma or other illnesses. The application programs and back end functionality for testing including data storage, analysis and scoring. The processor 1040 executes the program instructions to implement the application software and method described herein. The mobile device may also include input output devices, such as a camera, an accelerometer, GPS, a microphone, a touchscreen and other devices which produce data or a real time stream of data that are used in the concussion testing to caputre audio, video and other data. The mobile device may be a computer, laptop, mobile phone, pda, tablet or any other compiting device.

While particular embodiment have been illustrated and described, it will be understood that changes may be made to those embodiments without departing from the spirit and scope of the present invention.

1. A device having an application program for collecting user voice data and heartrate data interactively, comprising:
a memory, including program instructions, for collecting user's voice data and heartrate interactively; and
a processor coupled to the memory for executing the program instructions to interact with the users to collect their voice and heartrate data and meta information based on pre-defined questions from their devices.

2. The device according to claim 1, wherein the processor is configured to run on different operating systems including Windows, iOS, and Android.

3. The device according to claim 2, wherein the processor is configured to further execute program instructions to perform tasks for which subjects take (a) lung reserve assessment with counting test, (b) speech assessment, (c) deep breathing assessment, (d) egophony screening test for pneumonia, and (e) cough pattern recognition test.

4. The device according to claim 3, wherein the program instructions stored in the memory further include program instructions to provide user interface for labelling and tagging data with guideline from medical doctors.

5. The device according to claim 4, wherein the program instructions stored in the memory further include program instructions for machine learning using multimodal voice and heartrate data collected from the current system and relevant data from elsewhere.

6. The device according to claim 5, wherein the program instructions stored in the memory further include program instructions to perform ASR, VAD, spectral analyses, LPC analyses, voice pitch and voice formants and voice intensity analyses, heartrate estimation, and machine learning.

7. The device according to claim 6, wherein the program instructions stored in the memory further include program instruction to perform the following analyses:
(a) using ASR to recognize counting words, analyze the highest count a patient reached and a total duration of the counting;
(b) using ASR to analyze reading speech by the patient and analyze the subject's speaking rate, breaths per minute, PSD, pitch, formants, inter-word breath, and intra-word breath;
(c) using VAD to detect inhale, pause, and exhale and analyze the breath duration, breath PSD, and pause duration;
(d) using ASR to recognize the patient's /E/ and /A/ speech and analyze the closeness of the two sounds against the patient's baseline and a general normal baseline; and
(e) using VAD to detect a patient's cough and compare the patient's cough to the patient's own baseline cough, and classify the cough into specific categories (wet cough versus dry cough).

8. The device according to claim 7, wherein the program instructions stored in the memory further include a program to run in different languages, including at least English and Spanish, to facilitate access.

9. The device according to claim 7, wherein the program instructions stored in the memory further include program instructions to conduct analyses using multimodal voice and heartrate data and machine learning, to summarize the evaluation results, and to provide a risk score and report to review and provide to doctors.

10. The device according to claim 7, wherein the program instructions stored in the memory further include program instructions to evaluate reliability of the voice-based and image-based heartrate data in VLFAA.

11. The device according to claim 7, wherein the program instructions stored in the memory further include program instructions to further collect meta data through voice on the subject's age, gender, condition, medical history, and activities before testing.

12. The device according to claim 11, wherein the program instructions stored in the memory further include program instructions to perform sentiment analysis to access the subject's anxiety level.

13. A mobile device for diagnosing respiratory disease, comprising:
a memory storing at least one application program capable of executing a plurality of tests each having a test protocol, the application program including program instructions;
a microphone and speaker;
a display;
a processor coupled to the memory, the display, the microphone and the speaker, the processor configured to execute the program instructions in order to: (i) instruct the user to perform an action including at least breathing, coughing, and reading text; (ii) activating the microphone to record the user following each instruction for each of the plurality of test protocols in respective recordings; (iii) analyzing and scoring the audio as according to criteria specific to each test protocol that are based on spectral data associated with the respective recordings; and (iv) determining a composite score for presence or absence of the disease based on the individual scores; (v) storing data, the test scores and the composite scores.

14. The mobile device according to claim 13, wherein the processor is configured to instruct the user to perform each test according to its respective test protocol using the display of the mobile device.

15. The mobile device according to claim 13, wherein the processor is configured to instruct the user to perform each test according to its respective test protocol using the speaker of the mobile device.

16. The mobile device according to claim 13, wherein the mobile device further includes a network interface and wherein the processor is further configured exchange data with a remote database and server to store data generated during (i)-(v) on the server and in the database.

17. The mobile device according to claim 16, wherein the server includes a processor and is configured to determine individual test scores and an aggregate score based on the data generated by the mobile device.

18. The mobile device according to claim 13, wherein the processor determines each breath that the user takes based on a spectral analysis of the users' performance of each test.

19. The mobile device according to claim 18, wherein the processor determines each breath that the user takes based on a spectral analysis of the users performance of each test as compared to a representative control.

20. The mobile device according to claim 18, wherein the processor determines each breath that the user takes based on a spectral analysis of the users performance of each test as compared to a prior baseline of the user.

21. The mobile device according to claim 13, wherein the processor determines PSD for the duration of a user's performance based on a spectral analysis of the users' performance of each test.

22. The mobile device according to claim 21, wherein the processor determines a score for at least some of the tests based on the PSD of the users' performance of the at least some of the tests as compared to a representative control.

23. The mobile device according to claim 21, wherein the processor determines a score for at least some of the tests based on the PSD of the users' performance of the at least some of the tests as compared to a prior baseline of the user.

24. The mobile device according to claim 13, wherein the processor is configured to determine a presence of a disease based on the user's voice uttering /A/ or /E/ when instructed and the outcome of ASR and confidence scores using the utterances.

25. The mobile device according to claim 13, wherein the scoring determines disease is more likely when the ASR fails to distinguish /A/ from /E/ or one or both of the confidence scores of the ASR determining /A/ or /E/ is low.
